(19) United States
(12) Patent Application Publication
(10) Pub. No.: US 2007/0293671 A1
(43) Pub. Date: Dec. 20, 2007

(54) Process for the Preparation of Risperidone

(76) Inventors: Laszlo Czibula, Budapest (HU); Peter Turcsanyi, Budapest (HU); Krisztina Feher, Budapest (HU); Ferenc Sebok, Mezokovacshaza (HU); Gyorgy Szabo, Budapest (HU); Eva Werkne Papp, Budapest (HU)

Correspondence Address: K.F. ROSS P.C., 5683 RIVERDALE AVENUE, SUITE 203 BOX 900, BRONX, NY, 10471-0900, US

(21) Appl. No.: 11/631,980

(22) PCT Filed: Jul. 6, 2005
(86) PCT No.: PCT/HU05/00072

§ 371 Date: Jun. 14, 2007

(30) Foreign Application Priority Data
Jul. 8, 2004 (HU) P0401379

(51) Int. Cl. C07D 239/70

(52) U.S. Cl. 544/282

(57) ABSTRACT

The invention relates to a process for the preparation of risperidone (chemical name: 3-[2-[4-(6-fluoro-1,2-benzisoxazole-3-yl)-1piperidmyl]ethyl-2-methyl-6,7,8,9-terahydro-4H-pyrido[1,2-a]pyrimidine-4-one) of the formula (I) by reacting 3-(2-chloroethyl)-2-methyl-6,7,8,9-tetrahydro-4H-pyrido[1,2-a]pyrimidine-4-one of the formula (II) and 6-fluoro-3-(4-piperidinyl)-1,2-benzisoxazole of the formula (III), in which the reaction is carried out in dry methanol solvent under pressure, at a temperature between 65 and 90° C., the product is recovered by using a methanol/water mixture of specified ratio and if desired is recrystallized from an alcohol.

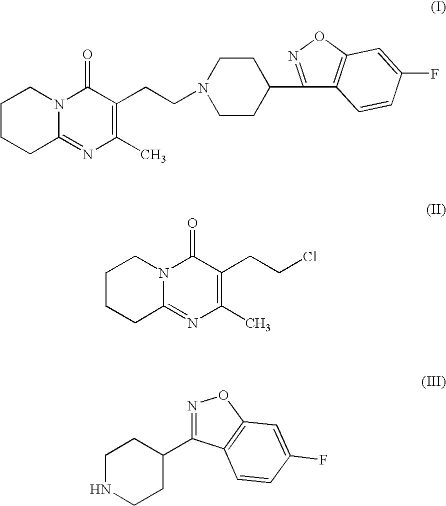

The invention relates to a process for the preparation of risperidone (chemical name: 3-[2-[4-(6-fluoro-1,2-benzisoxazole-3-yl)-1-piperidinyl]ethyl-2-methyl-6,7,8,9-terahydro-4H-1-pyrido[1,2-a]pyrimidine-4-one) of the formula (I) 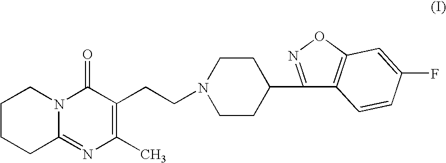
by reacting 3-(2-chloroethyl)-2-methyl-6,7,8,9-tetrahydro-4H-pyrido[1,2-a]pyrimidine-4-one of the formula (II) 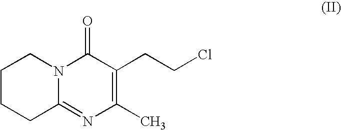
and 6-fluoro-3-(4-piperidinyl)-1,2-benzisoxazole of the formula (III), 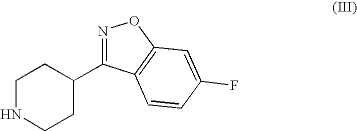
in which the reaction is carried out in dry methanol solvent under pressure, at a temperature between 65 and 90° C., the product is recovered by using a methanol/water mixture of specified ratio and if desired is recrystallized from an alcohol.

The risperidone has combined serotonin (5-HT₂) and dopamine (D₂) receptor antagonist effects (it is an antipsychotic compound) and plays an important role in the treatment of schizophrenia.

For the preparation of the risperidone several chemical processes have been developed, of which the syntheses using 3-(2-substituted ethyl)-2-methyl-6,7,8,9-tetrahydro-4H-pyrido[1,2-a]pyrimidine-4-one of the general formula (IV), 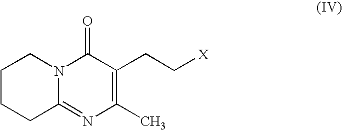
—wherein X stands for a halogen atom—, and 6-fluoro-3-(4-piperidinyl)-1,2-benzisoxazole of the formula (III) are preferred for industrial application.

According to the HU 195.793 Hungarian patent specification (Janssen Pharmaceutica) the risperidone is prepared from 3-(2-chloroethyl)-2-methyl-6,7,8,9-tetrahydro-4H-pyrido[1,2-a]pyrimidine-4-one of the formula (II) and an intermediate of the formula (III) in an inert solvent, such as dimethylformamide, in the presence of catalytic amount of sodium iodide. The crude product obtained is crystallized from a mixture of dimethylformamide and 2-propanol to give the product with a total yield of 46%.

According to WO 01/85731 published international patent application the same starting materials are reacted in water and the product obtained is recrystallized from dimethylformamide to give the risperidone with a yield of 73%. While in the reaction application of the carcinogenic dimethylformamide is avoided, recrystallization of the crude product is carried out from dimethylformamide. When the reaction is performed in water, the reactant of the formula (II) in the alkaline aqueous medium may undergo hydrolysis forming a hydroxyethyl derivative of the formula (V), 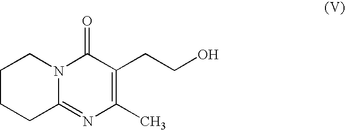
which appears as a considerable amount of contamination in the crude product. A further source of impurity is the starting benzisoxazole derivative of the formula (III), a part of which remains unreacted due to the hydrolytic loss of the compound (II). Another drawback of this process is that while both the starting material of the formula (II) and formula (III) are marketed as stable hydrochloride salts, they are used in the reaction in the form of bases which are susceptible to decomposition. The reason for this is that when the reaction is performed in aqueous medium it is strictly necessary to use said compounds in the base form; at least in our experiments when the hydrochlorides of the compounds of the formulae (II) and (III) were reacted in the presence of an alkali carbonate, a sticky mass was obtained which couldn't be stirred and was difficult-to-manage, particularly at industrial scale. Consequently, said hydrochloride salts first should be converted into the corresponding bases in an additional step which causes a substantial increase in the production costs. Besides these technological and economical problems there is also an environmental one: since the recrystallization is carried out from the carcinogenic dimethylformamide, the mother liquor obtained requires a special, environmentally acceptable work up.

In a process disclosed in WO 02/14286 (TEVA) published international patent application the compounds of the formulae (II) and (III) are reacted in a solvent different from the above (acetonitrile, 2-propanol, isobutanol, methyl ethyl keton, etc.) and the crude product is then recrystallized. The yield, however, even at best is below 63% and the product is rather contaminated.

Our aim was to provide a process lacking the disadvantages of the previous processes, i.e. to obtain the end-product in good yield and in the required drug-purity.

In the course of our experiments we have surprisingly found that when the reaction of the chloroethyl derivative of the formula (II) and the compound of the formula (III) is performed in dry methanol in a pressure vessel, in the presence of an acid binding agent, at 65-90° C., the product obtained is easy-to-stir during the whole reaction time independently of that the reactants are in the form of bases or in the form of hydrochloride salts, as they are marketed. The reaction takes 4-4.5 hours, which is shorter than that is generally disclosed in technical literature and the yield is higher than 90%. Further, it has been found that when the isolation of the product is done in a methanol-water mixture of specified ratio and the product after filtration is washed with ion exchanged water to remove inorganic salts, the purity of the risperidone quite unexpectedly is higher than 99%. In the course of the reaction no hydrolysis has been observed, consequently the product contains neither compound (V) nor other hydrolytic side products as impurity; i.e. there is no need for additional purification step. Another advantage is that product colouration—which may occur via oxidation—is also repressed.

Advantages of the process according to this invention are as follows: the reactants can not only be used as bases, but directly as they are available in the market, in the form of salts, when there is no need to convert them into the corresponding bases in a costly separate step; the reaction under pressure takes a short time (4-4.5 hours) and gives the product with a yield of 93% (known processes go with 46-73% yields); the crude product is obtained after washing with water in high purity (99%); a possible recrystallization doesn't cause environmental problems, since no dimethylformamide is used.

The object of the invention is a process for the preparation of risperidone (chemical name: 3-[2-[4-(6-fluoro-1,2-benzisoxazole-3-yl)-1-piperidinyl]ethyl-2-methyl-6,7,8,9-terahydro-4H-pyrido[1,2-a]pyrimidine-4-one) of the formula (I) 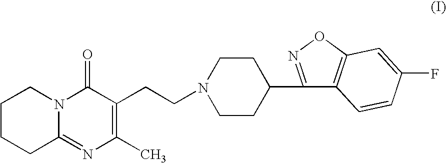
by reacting 3-(2-chloroethyl)-2-methyl-6,7,8,9-tetrahydro-4H-pyrido[1,2-a]pyrimidine-4-one of the formula (II) 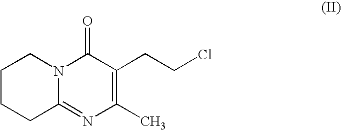
and 6-fluoro-3-(4-piperidinyl)-1,2-benzisoxazole of the formula (III), 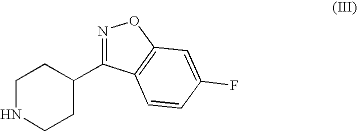
in which the reaction is carried out in dry methanol solvent under pressure, at a temperature between 65 and 90° C., the product is recovered by using a methanol/water mixture of specified ratio and if desired is recrystallized from an alcohol.

In a preferred embodiment of the invention the reaction is performed at 70-75° C., in the isolation step the methanol:water ratio by weight is adjusted to be from 1:0.8 to 1:1.2 and the recrystallization optionally is carried out from 2-propanol.

The purity of the risperidone (chemical name: 3-[2-[4-(6-fluoro-1,2-benzisoxazole-3-yl)-1-piperidinyl]ethyl-2-methyl-6,7,8,9-terahydro-4H-pyrido[1,2-a]pyrimidine-4-one) of the formula (I) was determined by HPLC as follows:
Column: Hypersil BDS C18, 100×4.6 mm ID, 3 μm
Eluent: A: 70% 5.0 g/l NH₄OAc+30% methanol
   B: 100% methanol

Gradient:

| Time | | | Flow rate |
|---|---|---|---|
| (min) | A % | B % | (ml/min) |
| 0 | 100 | 0 | 1.5 |
| 15 | 43 | 57 | 1.5 |
| 25 | 43 | 57 | 1.5 |
| 25.1 | 100 | 0 | 1.5 |
| 30 | 100 | 0 | 1.5 |

Detection: 260 nm, 30 min
Temperature: 25° C.
Injected volume: 10 μl
Sampling: 10 mg/ml, methanol
Approximative retention time: 10 min (risperidone)

The invention is illustrated by following non-limiting Examples.

### EXAMPLE 1

Preparation of the risperidone of the formula (I) from the hydrochlorid salt of 3-(2-chloroethyl)-2-methyl-6,7,8,9-tetrahydro-4H-pyrido[1,2-a]pyrimidine-4-one of the formula (II) and the hydrochloride salt of 6-fluoro-3-(4-piperidinyl)-1,2-benzisoxazole of the formula (III).

In a pressure vessel into a mixture of 13.3 g (0.052 mol) of 6-fluoro-3-(4-piperidinyl)-1,2-benzisoxazole hydrochloride, 15.0 g (0.057 mol) of 3-(2-chloroethyl)-2-methyl-6,7,8,9-tetrahydro-4H-pyrido[1,2-a]pyrimidine-4-one hydrochloride, 20.67 g of dry sodium carbonate and 200 ml of dry methanol nitrogen is introduced and the mixture is stirred for 4-4.5 hours at 73-75° C. Then the pressure is brought to atmospheric level, the mixture is concentrated to about 150 g, 100 ml of ion exchanged water is added, then the mixture is cooled to a temperature between 0° C. and 5° C. and filtered. To the filter cake 100 ml of ion exchanged water is added, stirred for an hour at 23-25° C. and filtered. The crystals are washed with ion exchanged water (3×20 ml), filtered and dried at a temperature below 60° C. to yield 20.0 g of risperidone (93.6% based on the starting benzisoxazole derivative).

Mp: 171-172° C.; purity is at least 99%, determined by HPLC

### EXAMPLE 2

Preparation of risperidone of the formula (I) from 3-(2-chloroethyl)-2-methyl-6,7,8,9-tetrahydro-4H-pyrido[1,2-a]pyrimidine-4-one and 6-fluoro-3-(4-piperidinyl)-1,2-benzisoxazole bases of the formulae (II) and (III).

Starting from 11.45 g of 6-fluoro-3-(4-piperidinyl)-1,2-benzisoxazole, 12.45 g of 3-(2-chloroethyl)-2-methyl-6,7,8,9-tetrahydro-4H-pyrido[1,2-a]pyrimidine-4-one and 11.8 g of dry sodium carbonate the same method as described in Example 1 is followed, to give 19.8 g (92.8%) of risperidone.

Mp: 171-172° C.; purity is at least 99%, determined by HPLC

1. A process for the preparation of risperidone (chemical name: 3-[2-[4-(6-fluoro-1, 2-benzisoxazole-3-yl)-1-piperidinyl]ethyl-2-methyl-6,7,8,9-terahydro-4H-pyrido[1,2-a]pyrimidine-4-one) of the formula (I) 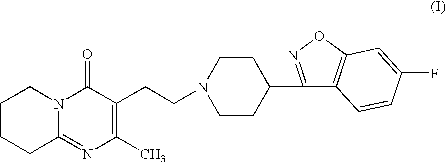
by reacting 3-(2-chloroethyl)-2-methyl-6,7,8,9-tetrahydro-4H-pyrido[1,2-a]pyrimidine-4-one of the formula (II) 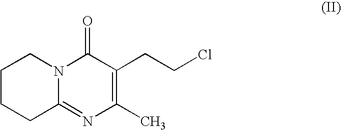
and 6-fluoro-3-(4-piperidinyl)-1,2-benzisoxazole of the formula (III), 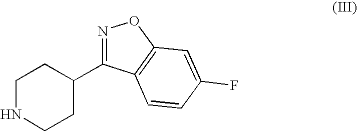
in which the reaction is carried out in dry methanol solvent under pressure, at a temperature between 65 and 90° C., the product is recovered by using a methanol/water mixture of specified ratio and if desired is recrystallized from an alcohol.

2. A process according to claim 1, characterized in that the reaction is carried out at 70-75° C.

3. A process according to claim 1, characterized in that in the isolation step the methanol:water ratio by weight is within the range from 1:0.8 to 1:1.2.

4. A process according to claim 1, characterized in that if desired recrystallization is carried out from 2-propanol.
